(19) United States
(12) Patent Application Publication
(10) Pub. No.: US 2010/0272604 A1
(43) Pub. Date: Oct. 28, 2010

(54) Radial Design Oxygenator with Heat Exchanger and Integrated Pump

(75) Inventors: Walt L. Carpenter, Minneapolis, MN (US); Kevin McIntosh, Brooklyn Park, MN (US)

Correspondence Address: Medtronic CardioVascular, Mounds View Facility South, 8200 Coral Sea Street N.E., Mounds View, MN, 55112, US

(73) Assignee: Medtronic, Inc., Minneapolis, MN (US)

(21) Appl. No.: 12/717,648

(22) Filed: Mar. 4, 2010

Related U.S. Application Data
(63) Continuation-in-part of application No. 12/428,689, filed on Apr. 23, 2009.

(51) Int. Cl. A61M 1/00; A61M 1/36

(52) U.S. Cl. 422/45; 422/46

(57) ABSTRACT

Disclosed is an apparatus for oxygenating and controlling the temperature of blood in an extracorporeal circuit. The apparatus has an inlet and an outlet that is located radially outward from the inlet in order to define a flowpath through the apparatus. The apparatus comprises at least one integrated pump that is provided in a core of the apparatus and to which blood from a patient can be supplied through the inlet; a heat exchanger comprising a plurality of heat transfer elements that are arranged around the at least one integrated pump and between which blood from the at least one integrated pump can move radially outward; an oxygenator comprising a plurality of gas exchange elements that are arranged around the heat exchanger and through which blood from the heat exchanger can move radially outward; and an optional filter arranged around the oxygenator and through which blood from the oxygenator can move radially outward before exiting the apparatus through the outlet.

### CROSS-REFERENCE TO RELATED APPLICATION

This application is a continuation-in-part application of U.S. patent application Ser. No. 12/428,689, filed on Apr. 23, 2009, which is incorporated herein by reference in its entirety.

### BACKGROUND OF THE INVENTION

A cardiopulmonary bypass circuit (i.e., a heart-lung bypass machine) mechanically pumps a patient's blood and oxygenates the blood during major surgery. Blood oxygenators are disposable components of heart-lung bypass machines used to oxygenate blood. A typical commercially available blood oxygenator integrates a heat exchanger with a membrane-type oxygenator.

Typically, in a blood oxygenator, a patient's blood is continuously pumped through the heat exchanger portion prior to the oxygenator portion. A suitable heat transfer fluid, such as water, is pumped through the heat exchanger, separate from the blood but in heat transfer relationship therewith. The water is either heated or cooled externally of the heat exchanger. The heat exchanger is generally made of a metal or a plastic, which is able to transfer heat effectively to blood coming into contact with the metal or plastic. After blood contacts the heat exchanger, the blood then typically flows into the oxygenator.

The oxygenator generally comprises a so-called “bundle” of thousands of tiny hollow fibers typically made of a special polymeric material having microscopic pores. The blood exiting the heat exchanger then flows around the outside surfaces of the fibers of the oxygenator. At the same time, an oxygen-rich gas mixture, sometimes including anesthetic agents, flows through the hollow fibers. Due to the relatively high concentration of carbon dioxide in the blood arriving from the patient, carbon dioxide from the blood diffuses through the microscopic pores in the fibers and into the gas mixture. Due to the relatively low concentration of oxygen in the blood arriving from the patient, oxygen from the gas mixture in the fibers diffuses through the microscopic pores and into the blood. The oxygen content of the blood is thereby raised, and its carbon dioxide content is reduced.

An oxygenator must have a sufficient volumetric flow rate to allow proper temperature control and oxygenation of blood. A disadvantage of perfusion devices incorporating such oxygenators is that the priming volume of blood is large. Having such a large volume of blood outside of the patient's body at one time acts to dilute the patient's own blood supply. Thus, the need for a high prime volume of blood in an oxygenator is contrary to the best interest of the patient who is undergoing surgery and is in need of a maximum possible amount of fully oxygenated blood in his or her body at any given time. This is especially true for small adult, pediatric and infant patients. As such, hemoconcentration of the patient and a significant amount of additional blood, or both, may be required to support the patient. Therefore, it is desirable to minimize the prime volume of blood necessary within the extracorporeal circuit, and preferably to less than 500 cubic centimeters. One way to minimize the prime volume is to reduce the volume of the blood oxygenator. There are limits to how small the oxygenator can be made, however, because of the need for adequate oxygen transfer to the blood, which depends in part on a sufficient blood/membrane interface area.

The cells (e.g., red blood cells, white blood cells, platelets) in human blood are delicate and can be traumatized if subjected to shear forces. Therefore, the blood flow velocity inside a blood oxygenator must not be excessive. The configuration and geometry, along with required velocities of the blood make some perfusion devices traumatic to the blood and unsafe. In addition, the devices may create re-circulations (eddies) or stagnant areas that can lead to clotting. Thus, the configuration and geometry of the inlet port, manifolds and outlet port for a blood flow path is desired to not create re-circulations (eddies), while also eliminating stagnant areas that can lead to blood clot production.

Overall, there is a need for improved components of cardiopulmonary bypass circuits. Such improved components will preferably address earlier problematic design issues, as well as be effective at oxygenating and controlling the temperature of blood.

### SUMMARY OF THE INVENTION

The present invention overcomes the shortcomings of the prior art by providing an apparatus that is part of a cardiopulmonary bypass circuit and that oxygenates and controls the temperature of blood external to a patient using a design that allows blood to flow radially and sequentially through a pump, a heat exchanger, an oxygenator, and, optionally, a filter. The heat exchanger can be arranged around (e.g., concentrically about) a core comprising an integrated pump, and the oxygenator is arranged around (e.g., concentrically about) the heat exchanger, or vice versa. As blood is delivered into the core comprising the integrated pump, it is moved radially outward through both the heat exchanger and oxygenator, as well as the optional filter. A heat transfer medium is preferably supplied separately to the heat exchanger and an oxygen-containing gas medium is supplied separately to the oxygenator, with both media being supplied in directions generally transverse to the radial movement of the blood through the apparatus.

One advantage of the radial movement of blood from the integrated pump through both the heat exchanger and the oxygenator in the apparatus is that it increases the overall performance and efficiency of the apparatus. The radial design provides optimal distribution of blood over surface area used for gas and heat exchange. The radial flow also results in a low pressure drop within the apparatus.

In certain embodiments of the invention, the oxygenator is located around or downstream from the heat exchanger. Because gas solubility varies significantly with temperature, it is important that blood be oxygenated at the temperature at which it will enter the body. Heating the blood before oxygenating the blood, therefore, can be desirable.

The radial blood flow through both the heat exchanger and oxygenator decreases recirculation of blood and/or stagnant areas of blood, which reduces the chance of blood clots. In addition, the radial flow minimizes shear forces that would otherwise traumatize blood cells.

Another advantage of the apparatus is that the design eliminates certain components necessary in prior art devices, which in turn reduces the prime volume of blood necessary for the apparatus. The benefit of reducing prime volume is that a patient undergoing blood oxygenation is able to maintain a maximum possible amount of fully oxygenated blood in his or her body at any given time during surgery. This is especially important for small adult, pediatric and infant patients.

The apparatus also has improved manufacturability over other such apparatuses. The invention includes fewer necessary parts than other similar devices, which makes the apparatus easier and less expensive to manufacture.

An embodiment of the invention is an apparatus for oxygenating and controlling the temperature of blood in an extracorporeal circuit. The apparatus has an inlet and an outlet that is located radially outward from the inlet in order to define a flowpath through the apparatus. As discussed above, the apparatus comprises a core comprising an integrated pump to which blood from a patient can be supplied through the inlet; a heat exchanger comprising a plurality of heat transfer elements that are arranged around the integrated pump and between which blood from the integrated pump can move radially outward; and an oxygenator comprising a plurality of gas exchange elements that are arranged around the heat exchanger and through which blood from the heat exchanger can move radially outward, and optionally, a filter arranged around the oxygenator and through which blood from the oxygenator and heat exchanger can more radially outward before exiting the apparatus through the outlet.

In the embodiment described above, the plurality of heat transfer elements may be arranged concentrically about the integrated pump. The plurality of gas exchange elements may be arranged concentrically about the heat exchanger. The plurality of heat transfer elements may be wound on the integrated pump, and the plurality of gas exchange elements may be wound on the heat exchanger. The heat exchanger may be arranged around the integrated pump such that blood can move from the integrated pump to the heat exchanger without structural obstruction. The oxygenator may be arranged around the heat exchanger such that blood can move from the heat exchanger to the oxygenator without structural obstruction. The optionally filter may be arranged around the oxygenator such that blood can move from the oxygenator to the outlet without structural obstruction.

The integrated pump may be selected from the group of pumps that are capable of delivering outflow over a substantially 360 degree perimeter, e.g., a centrifugal pump, a diaphragm pump or a balloon pump. Alternatively, a pump that can be configured to achieve such flow distribution can be utilized. The integrated pump may have a central axis, and may pump blood radially outward to the heat exchanger in a substantially transverse direction to the central axis. In one example, the apparatus includes an integrated pump having a central axis, and blood may move radially outward from the integrated pump, oxygenator, and/or heat exchanger through all or substantially all of the 360 degrees around the central axis.

The plurality of heat transfer elements may include a lumen through which a fluid medium can be supplied in order to control the temperature of blood moving between the heat transfer elements. The plurality of heat transfer elements may be arranged such that movement of the fluid medium through the plurality of heat transfer elements is substantially transverse to the radially outward direction that blood can move between the plurality of heat transfer elements. The oxygenator may comprise a plurality of gas exchange elements that include lumens through which an oxygen-containing gas medium can be supplied in order to oxygenate blood moving between the plurality of gas exchange elements. The plurality of gas exchange elements may be arranged such that movement of the gas medium through the plurality of gas exchange elements is substantially transverse to the radially outward direction that blood may move between the plurality of gas exchange elements. As an option, the apparatus may further comprise a filter, for example, a filter through which blood can move before exiting the apparatus through the outlet. In one embodiment, the filter is arranged concentrically around the oxygenator and through which blood from the oxygenator may move in a radial outward direction before exiting the apparatus through the outlet.

The apparatus may further comprise a housing that retains the integrated pump, the heat exchanger and the oxygenator. The housing may include the inlet, which is in communication with the integrated pump. The housing may include the outlet, which is located radially outward from the oxygenator.

### BRIEF DESCRIPTION OF THE DRAWINGS

The invention will be further explained with reference to the appended Figures, wherein like structure is referred to by like numerals throughout the several views, and wherein:

FIG. 1 is a schematic drawing of a cardiopulmonary bypass circuit including an apparatus in accordance with the invention;

FIG. 2 is a schematic drawing of an apparatus, in accordance with the invention, showing blood, fluid medium and gas medium flow through the apparatus;

FIG. 3 is a cross-sectional view of an embodiment of an apparatus including an integrated pump, in accordance with the invention;

FIG. 4 is a cross-sectional view of one embodiment of a apparatus of the present invention having an alternative integrated pump and shown with a schematic view of a system into which the apparatus may be incorporated, in accordance with the invention;

FIG. 5 is a cross-sectional view of a core (with integrated pump not shown) illustrating one embodiment of a heat exchanger made of a plurality of wedges, and an oxygenator, in accordance with the invention;

FIG. 6 is a schematic view showing oxygenator fibers being wound on a heat exchanger in the early stage of the winding process, in accordance with the invention; and

FIG. 7 is a schematic representation of a winding apparatus for the method of winding oxygenator fibers, in accordance with the invention.

### DETAILED DESCRIPTION OF THE INVENTION

Prior to setting forth the invention, it may be helpful to an understanding thereof to set forth definitions of certain terms that will be used hereinafter:

The terms “comprises” and variations thereof do not have a limiting meaning where these terms appear in the description and claims.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, “a,” “an,” “the,” “at least one,” and “one or more” are used interchangeably.

As used herein, the term “or” is generally employed in its sense including “and/or” unless the content clearly dictates otherwise. The term “and/or” means one or all of the listed elements or a combination of any two or more of the listed elements. For example, “oxygenator, and/or heat exchanger” means oxygenator or heat exchanger or both oxygenator and heat exchanger.

As used herein, all numbers are assumed to be modified by the term “about” and preferably by the term “exactly.” Notwithstanding that the numerical ranges and parameters setting forth the broad scope of the invention are approximations, the numerical values set forth in the specific examples are reported as precisely as possible. All numerical value, however, inherently contain certain errors necessarily resulting from the standard deviation found in their respective testing measurements.

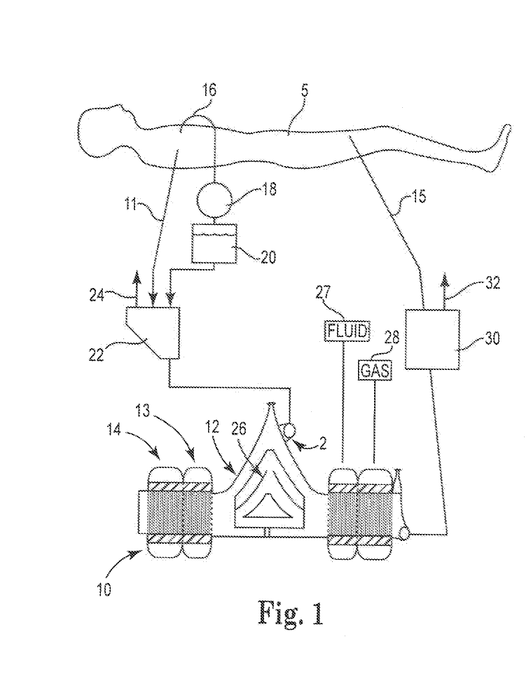

Turning now to FIG. 1, an exemplary cardiopulmonary bypass circuit is schematically illustrated, which includes an embodiment of an apparatus 10 in accordance with the invention. The circuit generally draws blood of a patient 5 during cardiovascular surgery through a venous line 11, oxygenates the blood, and returns the oxygenated blood to the patient 5 through an arterial line 15. Venous blood drawn from the patient through line 11 is discharged into a venous reservoir 22. Cardiotomy blood and surgical field debris are aspirated by a suction device 16 and are pumped by pump 18 into a cardiotomy reservoir 20. Once defoamed and filtered, the cardiotomy blood is also discharged into venous reservoir 22. Alternatively, the function of the cardiotomy reservoir 20 may be integrated into the venous reservoir 22. In the venous reservoir 22, air entrapped in the venous blood rises to the surface of the blood and is vented to the atmosphere through a purge line 24.

An integrated pump 26 is incorporated into the apparatus 10 and draws blood from the venous reservoir 22 through the apparatus 10 of the invention. Some exemplary types of integrated pumps 26 include, but are not limited to, centrifugal pumps, diaphragm pumps, and balloon pumps. Integrated pump 26 is described in more detail hereinbelow.

Apparatus 10 is configured such that blood is able to flow radially outward from the integrated pump 26 to a heat exchanger 13, preferably comprising a plurality of heat transfer elements that are located around the integrated pump 26. The plurality of heat transfer elements may be concentrically arranged about the integrated pump 26. The plurality of heat transfer elements may be wound or placed such that a space results between the heat exchanger 13 and the integrated pump 26. Preferably, there is minimal or no structural obstruction to blood flow between the integrated pump 26 and heat exchanger 13.

A heat transfer medium is supplied by a fluid supply 27 to the plurality of heat transfer elements and removed as indicated schematically. The fluid medium is preferably heated or cooled separately in the fluid supply 27 and is provided to the plurality of heat transfer elements in order to control the temperature of the blood flowing radially outward from the integrated pump 26 and between the heat transfer elements. Alternatively, the heat transfer medium may not be a fluid, but could be thermal energy that is conducted through the heat transfer elements in order to heat the blood.

Next, the blood moves radially outward from the heat exchanger 13 to an adjacent oxygenator 14, preferably comprising a plurality of gas exchange elements that are located around the heat exchanger 13. The plurality of gas exchange elements may be concentrically arranged about the heat exchanger 13. The plurality of gas exchange elements may be wound directly on the heat exchanger 13, or may be wound or placed such that a space or void results between the heat exchanger 13 and the oxygenator 14. Preferably, there is minimal or no structural obstruction to blood flow between the heat exchanger 13 and the oxygenator 14.

The oxygenator 14 is preferably a membrane oxygenator, and most preferably a hollow fiber oxygenator. Thus, the gas exchange elements are preferably fibers, although other such elements are also contemplated. An oxygen-containing gas medium is preferably supplied by gas supply 28 to lumens of the gas exchange elements and removed, as shown schematically. The oxygen-containing gas medium is provided to the oxygenator 14 in order to deliver oxygen to the blood flowing radially between the plurality of gas exchange elements, as well as to remove carbon dioxide.

The fluid and gas media and the blood moving through the apparatus 10 are preferably compartmentalized or kept separate, so as to not allow mixing. The direction of movement of the fluid and gas media through the heat exchanger 13 and oxygenator 14 of the apparatus 10 are preferably generally transverse to the direction of radial blood flow through the apparatus 10.

Oxygenated and temperature-controlled blood is collected after moving out of the oxygenator 14 of the apparatus 10, and preferably flows to an arterial filter 30 and then into the arterial line 15. The arterial filter 30 preferably traps air bubbles in the blood that are larger than about 20-40 micrometers where the bubbles can be removed through a purge line 32.

The circuit shown in FIG. 1 is exemplary, and it should be understood that the apparatus 10 of the invention may be incorporated into any suitable cardiopulmonary bypass circuit or other suitable extracorporeal system, for example.

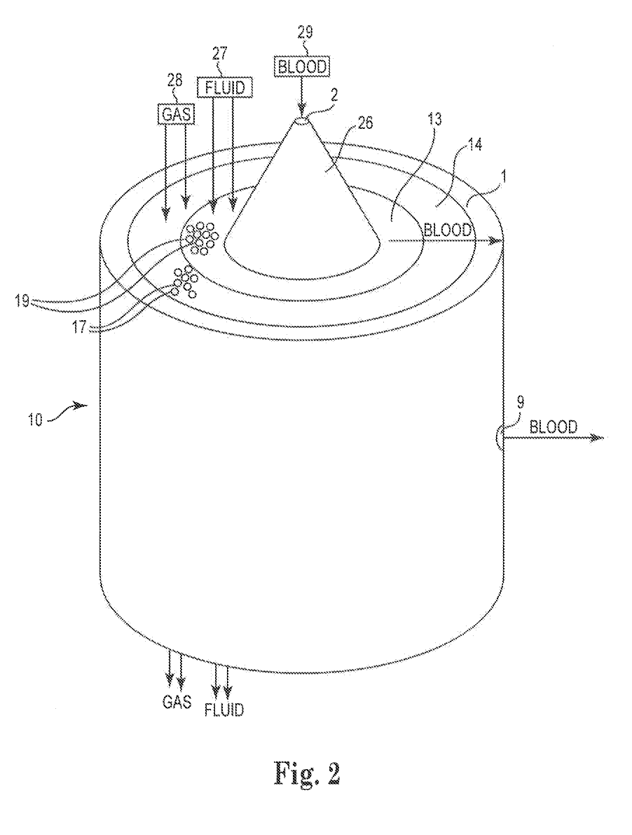

FIG. 2 is a schematic, perspective view of the apparatus 10 of the invention with flow of blood through the apparatus 10 and flow of fluid medium and gas medium into and out of the apparatus 10 indicated by arrows labeled as such. Pumped by integrated pump 26, blood from a patient enters blood inlet 2 from a blood supply 29 (e.g., a venous reservoir). The blood then sequentially moves radially outward from the integrated pump 26 into the heat exchanger 13 that is located around, and preferably arranged concentrically about, the integrated pump 26. In one embodiment, the blood moves continuously radially outward through substantially all of 360 degrees around the integrated pump 26 and evenly along substantially all of the length of the integrated pump 26. Sequentially, the blood moves radially outward from the heat exchanger 13 to and through the oxygenator 14 that is located around, and preferably arranged concentrically about, the heat exchanger 13. In one embodiment, the blood moves continuously radially outward through substantially all of 360 degrees around the heat exchanger 13 and the oxygenator 14. The oxygenated and temperature-controlled blood is then collected and exits the apparatus 10 preferably from an outlet port 9 in apparatus 10, and is returned to the patient through an arterial line (not shown). The apparatus 10 may include a housing, such as housing 1, wherein the blood is collected, for example, on an inner surface thereof (not shown), and through which blood is allowed to exit the apparatus 10 through outlet 9.

Blood circulated through apparatus 10 can be filtered before being returned to the patient, for example, in order to remove air bubbles. Thus, apparatus 10 optionally includes a filter through which oxygenated blood can flow through in a radially outward direction before exiting the apparatus and being returned to the patient. For example, the filter (not shown in FIG. 1) may be placed around the oxygenator 14, e.g., arranged concentrically around the oxygenator.

The heat transfer medium that is supplied to the heat exchanger 13 from a fluid medium supply 27 is heated or cooled externally to the apparatus 10. The fluid medium is supplied to lumens in a plurality of heat transfer elements 19 (only several of which are illustrated in FIG. 2) that comprise the heat exchanger 13. The heat transfer elements 19 conduct heat and either heat or cool the blood as the blood moves radially through the heat transfer elements 19 of the heat exchanger 13.

The gas medium that is supplied to the oxygenator 14 contains oxygen. The gas medium is delivered to lumens in a plurality of gas exchange elements 17 (only several of which are illustrated in FIG. 2) that comprise the oxygenator 14. The gas exchange elements 17 are preferably hollow fibers that are microporous in nature, which allows oxygen in the gas exchange elements 17 to diffuse through micropores into blood flowing between the gas exchange elements 17 and also allows carbon dioxide to diffuse from the blood into the gas medium in the gas exchange elements 17 and be removed from the blood.

The purpose of the radial design of the apparatus 10 is to allow for substantially continuous radial flow of blood through the apparatus 10. The radial flow design is beneficial because it optimizes distribution of the blood to the surface area for heat and oxygen exchange, which makes the design more efficient. Also, substantially continuous radial flow decreases the recirculation of blood and stagnant areas of blood with the apparatus, which decreases the chances of blood clotting. In addition, the design decreases shear forces on the blood, which can cause damage to blood cells. The radial design also decreases the prime volume of blood necessary compared to other such devices, which is beneficial for smaller patients, including children and small adults.

In order for the apparatus 10 to work efficiently, the gas medium, fluid medium and blood are compartmentalized or separated in the apparatus 10.

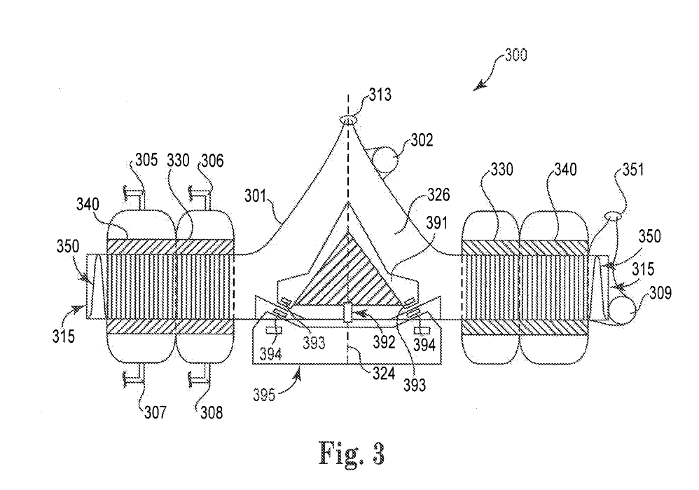

One embodiment of the present invention is depicted in FIG. 3, which is a cross-sectional view of an apparatus 300. The cross-sectional view in FIG. 3 shows details that may be incorporated into the apparatus of the invention. The apparatus 300 comprises the integrated pump 326, a heat exchanger 330, an oxygenator 340 and a filter 350. The integrated pump 326 is preferably located at or near the center of the apparatus 300. The heat exchanger 330 is positioned adjacent to the integrated pump 326, e.g., arranged concentrically around, and the oxygenator 340 adjacent to the heat exchanger 330, e.g., arranged concentrically around.

The heat exchanger 330 preferably comprises a bundle or plurality of hollow, heat transfer elements, which may be fibers, tubes, capillaries, compartments, etc. In one embodiment, the heat transfer elements comprise a conductive polymer or a metal. Various shapes of heat transfer elements are contemplated by the invention. One exemplary material for the heat transfer elements is a hollow fiber, for example, polyethylene terephthalate such as a HEXPET™ heat exchange capillary commercially available from Membrana, Charlotte, N.C., U.S.A.

In one example, the heat exchange capillary is provided in a mat comprising two layers of hollow capillaries that are made of polyethylene terephthalate (PET) with the two layers being angled with respect to one another. Preferably, the capillaries in one layer are at about a 15 degree angle or bias from normal. Thus, if two layers of the material are layered so that they have opposing biases, the net resulting degree of bias for the capillaries between the two layers is 30 degrees. A purpose for the opposing biases is to prevent any nesting of the capillaries between the two layers, which could result in increased resistance to blood flow and undesirable and unpredictable shear on the blood flowing there through (i.e., between the fibers). Other materials are contemplated by the present invention, however. The purpose of the heat transfer elements of the heat exchanger 330 is to transfer heat to or from the fluid medium running there through to or from the blood that flows between the heat transfer elements.

The heat transfer elements of the heat exchanger 330 are located around the integrated pump 326, and may be, for example, tightly wound or wrapped concentrically about the integrated pump 326. Also, the heat transfer elements may be located such that there is minimal or no structural obstruction between the integrated pump 326 and the heat exchanger 330. Alternatively, the heat exchanger may comprise heat transfer elements that are pre-arranged in a woven, mat or fabric-like arrangement that may be assembled around the integrated pump 326, and either in direct contact with the integrated pump 326 or such that there is minimal or no structural obstruction to blood flow between the integrated pump 326 and the heat exchanger 330.

The heat exchanger 330 may either heat or cool the blood flowing through the apparatus 300. Because hypothermia may be used during cardiac surgery (especially in infant and pediatric surgeries) to reduce oxygen demand, and because rapid re-warming of the blood can produce gaseous emboli, the heat exchanger 330 is generally used to gradually re-warm blood and prevent emboli formation.

The heat transfer medium used in the heat exchanger 330 may comprise water or other suitable fluids. The heat exchanger 330 may comprise hot and cold tap water that is run through the plurality of heat transfer elements. Preferably, however, a separate heater/cooler unit with temperature-regulating controls is used to heat or cool the fluid medium outside of the apparatus 300, as necessary to regulate the temperature of the blood flowing between the heat transfer elements. As another alternative, a heat transfer means other than a fluid is possible. For example, thermal energy may be supplied to the heat transfer elements rather than a fluid.

Alternative configurations for heat transfer elements of the heat exchanger 330 are possible. If the heat transfer elements are wound on the integrated pump 326, for example, the elements of the heat exchanger 330 may preferably be surrounded by an elastic band or some other thin, flexible, horizontally extending woven interconnect (not shown) in order to hold them together and in place. After winding, ends of the heat transfer elements that are located near the ends of the combination of the integrated pump 326 and heat exchanger 330 are cut to allow the heat exchange fluid medium to enter lumens in the heat transfer elements.

The integrated pump 326 depicted in FIG. 3 is a centrifugal blood pump, which generally comprises a rotator 391 that rotates with respect to stator 392 in order to pump blood through apparatus 300. Rotation is caused by magnets 393 located in the rotator 391 interacting with magnets 394 in drive mechanism 395, which is external to apparatus 300. A particular centrifugal blood pump that may be used in the invention is the Bio-Pump™ Blood Pump, available from Medtronic™, Inc., located in Minneapolis, Minn., U.S.A. Other pumps are contemplated by the invention, however, and the particular type of pump shown in FIG. 3 is exemplary. For example, pumps that are capable of delivering outflow over a substantially 360 degree perimeter may be used. Alternatively, a pump that can be configured to achieve such flow distribution can be utilized, such as a diaphragm pump or a balloon pump, may be used. In addition, more than one pump may be used in order to achieve desired blood flow through the apparatus.

Pumps are preferably chosen that are able to provide continuous, radial flow. However, it is contemplated that alternative types of pumps and combinations of pumps may be used with design adjustments being made in the apparatus or system into which the apparatus is incorporated.

The purpose of the integrated pump 326 being located in the core or center of apparatus 300 is to push blood entering through blood inlet port 302 radially outward through the remainder of apparatus 300. The arrangement of the integrated pump 326, heat exchanger 330 and oxygenator 340 allows blood from a patient to enter the apparatus 300 at blood inlet port 302 and move radially outward through the apparatus 300. As an example, the integrated pump 326 propels the blood radially outward through substantially all of 360 degrees surrounding a central axis 324 that extends longitudinally through pump 326. The blood then flows sequentially and radially from the pump 326, into the heat exchanger 330 and then into the oxygenator 340. Optionally, the blood also flows through the filter 350 prior to exiting the apparatus 300 at outlet port 309.

There are two air purge ports that may be included in apparatus 300. One of the ports is purge port 313, which is located in the area of the integrated pump 326. The second port 351 is located in the filter 350 in order to purge any air bubbles that are filtered out of the blood prior to being returned to the patient.

Filter 350 may be formed from any suitable filtration medium, and may be arranged in any suitable manner, so as to provide filtration as the blood moves through the filter in a radially outward direction through the apparatus as described herein. For example, filter 350 can be arranged concentrically around the oxygenator. Blood moves through the filter in a radially outward direction in substantially all of 360 degrees around the central axis of the pump. Moreover, the filter 350 is arranged in such a manner so as to minimize any structural obstruction to the blood as it moves through the apparatus.

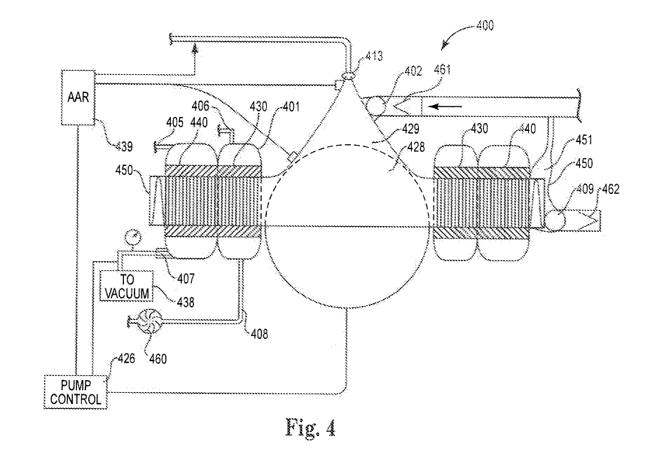

FIG. 4 depicts apparatus 400 including an alternative type of integrated pump, in particular, an integrated diaphragm pump 429. The figure also includes a schematic representation of a system into which the apparatus 400 may be incorporated. In general, the foregoing description of apparatus 300 also applies regarding FIG. 4, with the exception of the integrated diaphragm pump 429. The integrated diaphragm pump 429 shown pumps blood by using a diaphragm 428 that moves up and down, which is different from centrifugal force used in the integrated pump 326 of the embodiment in FIG. 3.

Referring again to FIG. 3, it is contemplated that the oxygenator 330 may be formed by following a method for helically winding continuous, semi-permeable, hollow fiber directly on the heat exchanger so as to eliminate or minimize any structural obstruction to blood flow between the heat exchanger 330 and the oxygenator 340. As an alternative, the oxygenator may be wound upon an intermediary component, e.g., a mandrel, so as to provide minimal structural obstruction to blood flow between the heat exchanger 330 and the oxygenator 340.

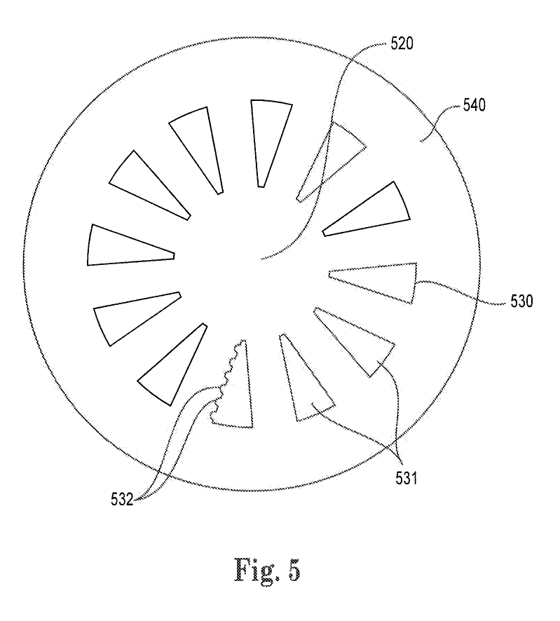

As discussed above, the heat exchanger may comprise any suitable material. Furthermore, heat exchanger may comprise any suitable configuration. For example, FIG. 5 shows a cross-sectional view of a core 520 (integrated pump not shown), a heat exchanger 530 and an oxygenator 540, which are components of an embodiment of the apparatus of the invention. In the embodiment, the plurality of heat transfer elements of the heat exchanger 530 comprise a plurality of wedges 531 that are configured and positioned such that blood flowing from the core 520 flows radially outward between the wedges 531. A fluid medium runs through lumens in the wedges 531 in order to transfer heat to or from the blood. The wedges 531 of heat exchanger 530 preferably comprise a metal or a conductive polymer. Preferably, the wedges 531 may be made using an extrusion process.

As another alternative, the wedges may include ribs or ridges 532, or other protrusions, on the surfaces that contact blood. The purpose of the ribs or ridges 532 are to both increase the surface area for heat transfer and to promote mixing to increase convective heat transfer to or from the blood. If an extrusion process is used to make the wedges 531, then the ribs or ridges 532 may be formed during the extrusion process. However, the ribs or ridges 532, or any other protrusions, located on the wedges 531, may alternatively be placed on the surface of the wedges 531 by other means after the wedges 531 are already formed.

Alternatively, any suitable material and/or configuration for the heat exchanger that preferably allows the heat exchanger to regulate temperature, have radial flow around substantially all of 360 degrees are contemplated by the invention.

Turning again to FIG. 3, after blood flows through the heat exchanger 330, it moves sequentially and radially outward to and through the oxygenator 340 that is arranged around the heat exchanger 330. The oxygenator 340 may concentrically surround the heat exchanger 330. Also, the oxygenator 340 may be wound on the heat exchanger 330. Preferably there is minimal or no structural obstruction to blood flow between the heat exchanger 330 and the oxygenator 340. The direction of blood flow is preferably maintained as radial, and does not substantially change through the heat exchanger 330 and the oxygenator 340.

FIG. 3 also depicts gas inlet port 305 and exit at gas outlet port 307. Preferably, the oxygenator 340 is a membrane oxygenator comprising a plurality of gas exchange elements, e.g., microporous hollow fibers. The blood flowing radially outward from the heat exchanger 330 moves radially between the gas exchange elements that comprise the oxygenator 340. Preferably, a bundle or plurality of hollow fibers are used for gas exchange elements and are made of semi-permeable membrane including micropores. Preferably, the fibers comprise polypropylene, but other materials are also contemplated by the invention. Any suitable microporous and/or gas permeable fiber may be used as the gas exchange elements of the oxygenator 340 of the invention.

An oxygen-containing gas medium is provided through the gas exchange elements, comprising the oxygenator 340. An oxygen-rich or -containing gas mixture supplied via the gas inlet 305 travels down through the interior or lumens of the gas exchange elements. Certain gases are able to permeate the gas exchange elements. Carbon dioxide from the blood surrounding the gas exchange elements diffuses through the walls of the gas exchange elements and into the gas mixture. Similarly, oxygen from the gas mixture inside the gas exchange elements diffuses through the micropores into the blood. The gas mixture then has an elevated carbon dioxide content and preferably exits the opposite ends of the gas exchange elements that it enters into and moves out of the apparatus 300 through the gas outlet 307. Although oxygen and carbon dioxide are preferably being exchanged, as described above, the invention also contemplates that other gases may be desired to be transferred.

Any suitable gas supply system may be used with the oxygenator 340 of the invention. For example, such a gas supply system may include flow regulators, flow meters, a gas blender, an oxygen analyzer, a gas filter and a moisture trap. Other alternative or additional components in the gas supply system are also contemplated, however.

Gas exchange elements of the oxygenator 340 are arranged around the heat exchanger 330, and preferably in a generally cylindrical shape. The gas exchange elements of the oxygenator 340 can be wound directly on the heat exchanger 330. In one embodiment, in order to form the oxygenator 340, one long microporous fiber may be wound back and forth on the heat exchanger 330. After winding, the fiber is cut at a plurality of locations that are located near the ends of the combination of the heat exchanger 330 and oxygenator 340, which will allow the gas medium to enter the portions of the fiber.

Once again referring to FIG. 3, after blood has traveled radially outward through the apparatus 300, oxygenated blood having a desired temperature is preferably collected along an inner surface of the housing 301 surrounding the oxygenator 340. In one embodiment, a collection area (not shown) or space for collection is provided radially outward from the oxygenator 340 and inside the housing 301. Preferably, the blood in the collection area 315, which surrounds the oxygenator 340, moves along the inner surface of the housing 301 and then flows out of the apparatus 300 through a blood outlet port 309 that is in fluid communication with the collection area 313. Preferably, one outlet port 309 is present, as shown, however, it is also contemplated that there may be more than one outlet port 309.

As discussed above, apparatus 400 in FIG. 4 is shown incorporated into a system. The system shown preferably detects air in the system that is desired to be removed. When air is detected by an integrated active air removal (AAR) device 439, a pump control device 426, that is connected using a circuit line to pump 429, slows the pump 429 until the air is removed. The purpose of the system is to remove any air bubbles that are in the blood before the blood is returned to a patient. Preferably, the active air removal system 439 is incorporated into the top portion of the pump 429, and may alternatively be incorporated into a centrifugal pump (e.g., pump 427 in FIG. 4) with appropriate design adjustments. In one embodiment of the invention, a venous air removal device (VARD), for example, as disclosed in U.S. Pat. No. 7,335,334, is included in the system.

The apparatus 400 in FIG. 4 also includes one-way flow valves 461, 462, which are shown as duck-bill valves. Valve 461 is located at the blood inlet port 412, and valve 462 is located at blood outlet port 409. These one-way flow valves 461, 462 are necessary when using a diaphragm pump, such as pump 429. The purpose of such one-way flow valves is to ensure that the blood flows to the pump 429 of apparatus 400 at blood inlet 412 and out at blood outlet 409.

The system may also preferably include integrated safety features. For example, the system may include a means of assuring that both the gas side pressure and the fluid side pressure in the heat exchanger 430 and oxygenator 440, respectively, are maintained below the blood side pressure. In the system shown, the outlet port 408 on the heat exchanger 430 is under negative pressure. The outlet port 407 of the oxygenator 440 is connected to a vacuum in order to likewise pull the gas medium through the oxygenator 440 under negative pressure. These safety features are included to prevent air bubbles and fluids from being injected into a patient's blood supply as the internal pressures of the device fluctuate due to the action of the diaphragm pump.

Depicted in FIG. 3 is an exemplary housing 301 is shown that houses or encloses the core comprising the integrated pump 323, heat exchanger 130 and oxygenator 340 of the invention. The purpose of the design or configuration of the housing 301 is preferably to allow the gas medium, fluid medium and blood to be supplied to different, functional sections of the apparatus 300. The design shown in FIG. 3 prevents undesired mixture of the fluid medium, gas medium and blood. The configuration shown is exemplary, and other configurations are also contemplated by the invention. The housing 301 also provides inlets and outlets for the blood, the fluid medium used in the heat exchanger 330, and the gas medium used in the oxygenator 340.

The housing 301 is preferably made of a rigid plastic, the purpose of which is for this apparatus to be sturdy yet lightweight. One exemplary type of such a rigid plastic is a polycarbonate-ABS (Acrylonitrile Butadiene Styrene) alloy. Other suitable materials for the housing 301 are also contemplated by the invention.

The peripheral wall of the housing 301 preferably includes a blood outlet 309 for apparatus 300. The blood outlet 309 may comprise a tube or pipe leading away from the apparatus 300, which ultimately allows the blood to be returned to a patient (not shown). Other devices may be necessary in order to return the blood to the patient, but are not shown. An advantage of a single blood outlet 309, as shown, is that the outlet 309 does not substantially interfere with fluid flow dynamics of the radial blood flow in the apparatus 300. Other suitable locations and configurations for a blood inlet or outlet, however, are also contemplated.

The apparatus of the present invention may also include a temperature probe port, which is located such that the temperature of blood being returned to a patient may be monitored. The temperature probe port may include a temperature sensing or monitoring device, such as a thermister.

Apparatus 300 includes a gas outlet port 307. Tubing is preferably connected to the port 307 specifically when an anesthetic is included in the gas medium. If anesthetic is not used, however, gas is generally allowed to flow out of additional holes (not shown in figures) that are open to the air, and located in housing 301 and in communication with the oxygenator 340.

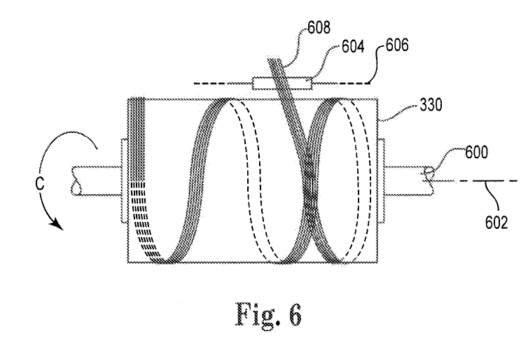

Generally, a winding apparatus, as shown in FIG. 6, may be used for fabrication of the device, which has a rotatable mounting member 600 having a longitudinal axis 602 and a fiber guide 604 adjacent said mounting member 600. The fiber guide 604 is adapted for reciprocal movement along a line 606 parallel to the longitudinal axis 602 of said mounting member 600 as the mounting member 600 rotates. The heat exchanger 330 is mounted for rotation on the rotatable mounting member 600. At least one continuous length of semi-permeable hollow fiber 608 (although more than one is shown) is provided where the hollow fiber is positioned by said fiber guide 604 and secured to said heat exchanger 330. The mounting member 600 is rotated and the fiber guide 604 is moved reciprocally with respect to the longitudinal axis 602 of the mounting member 600. Fiber or fibers 608 is or are wound onto said heat exchanger 330 to form the oxygenator 340 which extends radially outward relative to the axis of the mounting member 600 and which preferably has packing fractions which increase radially outwardly throughout a major portion of said oxygenator 340, thereby preferably providing a packing fraction gradient.

The foregoing method may involve two or more fibers 608 positioned by the fiber guide 604. The two or more fibers 608 are wound onto the heat exchanger 330, or an intermediary component, to form a wind angle relative to a plane parallel to the axis of the heat exchanger 330, tangential to the point at which the fiber is wound onto said heat exchanger 330 and containing said fiber 608.

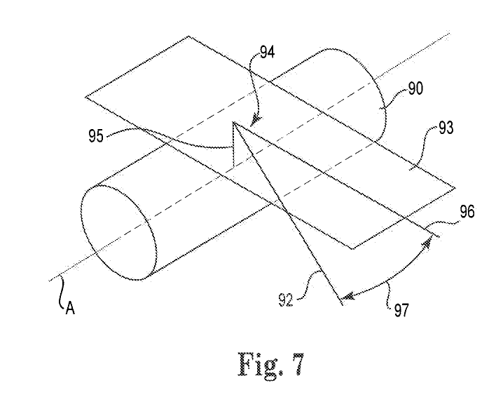

FIG. 7 illustrates the wind angle for a single fiber, but would apply as well for each of two or more fibers. Fiber 92 is contained in plane 93. Plane 93 is parallel to axis A of core 90. Plane 93 is tangential to point 94 at which fiber 92 is wound onto core 90. Line 95 is perpendicular to axis A and passes through point 94 and axis A. Line 96 is a projection into plane 93 of the normal line 95. Wind angle 97 is measured in plane 93 between projection line 96 and fiber 92. Alternatively, line 92 in tangential plane 93 is a projection into plane 93 from a fiber (not shown) which lies outside of plane 93.

The wind angle may be increased by increasing the distance through which the fiber guide moves during one rotation of the mounting thereby providing said increasing packing fraction. The wind angle may be decreased, increased or otherwise varied outside of the major portion of the bundle. The wind angle will be considered to have increased in the major portion of the bundle if on average it increases even though it may vary including decreasing.

The winding method may further involve tensioner means for regulating the tension of said fiber as it is wound. The tension of said fiber may be increased stepwise and continuously throughout a major portion of such winding thereby providing said increasing packing fraction. The fiber guide may be adapted to regulate the spacing between two or more fibers being simultaneously wound and the spacing may be decreased throughout a major portion of such winding thereby providing said increasing packing fraction.

The above-outlined procedure for spirally winding semi-permeable hollow fiber on a supporting core, such as on heat exchanger 330, for use in the blood oxygenator in accordance with the present invention is set forth in U.S. Pat. No. 4,975,247 (“247 patent”) at column 9, line 36 through column 11, line 63, including FIGS. 12 through 16A, all of which are incorporated herein by reference thereto for showing the following winding procedure. FIG. 16 of the '247 patent shows an alternative method for making a fiber bundle wherein a two-ply fiber mat 75 is rolled onto a core.

Guide 704 travels from the first end (left hand side of FIG. 7) of the heat exchanger 330 to the second end (right hand side of FIG. 7) where it decelerates. After decelerating, the guide 704 reverses direction and travels back to its starting position. After decelerating again and reversing direction, the guide begins its travel cycle anew. This reciprocal travel for guide 704 and the concurrent rotation of mounting member 700 on which the heat exchanger 330 has been mounted is continued, subject to the following described alteration, until an oxygenator 340 of desired diameter has been wound onto the heat exchanger 330.

As described more fully in columns 10-11 of the '247 patent, in the left-to-right travel of guide, a fiber ribbon was wound spirally around an extended support core (heat exchanger 330 in this invention) and the individual fibers in the ribbon were laid down in contact with the outer surfaces of support core ribs. In the known winding procedure, the core (heat exchanger 330 in this invention) is covered, except for the spacing between adjacent fibers and the distance between the sixth fiber of one ribbon and the first fiber of the next adjacent ribbon, when the fiber guide has traveled a sufficient number of traverses.

An exemplary pattern of winding the fibers of the oxygenator 340 is found on the Affinity™ Oxygenator (commercially available from Medtronic, Inc., Minneapolis, Minn., U.S.A.). However, alternatively, other methods and patterns of winding the oxygenator 340 fibers are also contemplated by the invention.

In making apparatus 300, once the oxygenator 340 is wound on the heat exchanger 330 (with or without any other components or space in between), ends of the heat transfer elements of the heat exchanger 330 and the gas exchange elements of the oxygenator 340 are preferably embedded in a potting composition in order to hold them together and in place in apparatus 300. The preferred potting material is polyurethane introduced by centrifuging and reacted in situ. Other appropriate potting materials or methods of potting the heat exchanger 330 and oxygenator 340 portions of the apparatus 300 are also contemplated by the invention.

Preferably, the potting composition is applied to both ends of the sets or pluralities of gas exchange elements and heat transfer elements that make up the oxygenator 340 and heat exchanger 330, which results in two regions of potted material. The potting material, however, covers the ends of the elements as well when applied in such a manner. Therefore, it is usually necessary to open the end of the heat transfer elements and gas exchange elements in order to allow communication with the gas and fluid media introduced to apparatus 300. Thus, once cured, a partial depth of the outer ends of the pottings are preferably sliced or cut (i.e., “guillotined”) in order to expose or open lumens of the heat transfer elements and gas exchange elements to allow gas and fluid media to be supplied to the lumens. Preferably, the potted ends are partially cut through in order to open the lumens of the heat transfer elements and gas exchange elements. The potted and cut ends of the heat transfer elements and gas exchange elements are then placed in the housing 301 such that the lumens of the heat transfer elements are in communication with the heat transfer medium and the lumens of the gas exchange elements are in communication with the oxygen-containing gas medium.

The fluid medium inlet 306 provides water, or another fluid medium, to the heat exchanger 330, in particular to one end of the plurality of heat transfer elements. The fluid medium is preferably heated or cooled outside of the apparatus 300, as necessary to regulate the temperature of blood flowing through the heat exchanger 330. The temperature of the blood can be monitored by a circuit (not shown) that includes a thermister or other temperature sensing device (not shown) mounted inside apparatus 300. After flowing through the heat exchanger 330, the fluid medium flows out of the heat exchanger 330 and the apparatus 300 through the fluid medium outlet 308.

After slicing the pottings and subsequent assembly of the apparatus 300, the lumens of the plurality of gas exchange elements of the oxygenator 340 are also able to be in communication with the gas inlet 305 and gas outlet 307. The oxygenator 340 is preferably supplied with a gas mixture rich in oxygen from a pressurized source (not shown) which is conveyed to the oxygenator 340 through gas inlet manifold 305.

Preferred embodiments of this invention are described herein, including the best mode known to the inventors for carrying out the invention. Of course, variations on those preferred embodiments will become apparent to those of ordinary skill in the art upon reading the foregoing description. The inventors expect skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. An apparatus comprising:
a core comprising an integrated pump to which blood from a patient can be supplied through an inlet;
a heat exchanger comprising a plurality of heat transfer elements that are arranged around the pump and between which blood from the pump can move radially outward; and
an oxygenator comprising a plurality of gas exchange elements that are arranged around the heat exchanger and through which blood from the heat exchanger can move radially outward before exiting the apparatus through an outlet.

2. The apparatus of claim 1, wherein the plurality of heat transfer elements are arranged concentrically about the pump.

3. The apparatus of claim 1, wherein the plurality of gas exchange elements are arranged concentrically about the heat exchanger.

4. The apparatus of claim 1, wherein the plurality of heat transfer elements are wound on the pump.

5. The apparatus of claim 1, wherein the plurality of gas exchange elements are wound on the heat exchanger.

6. The apparatus of claim 1, further comprising a filter arranged around the oxygenator and through which blood can move radially outward before exiting the apparatus through the outlet.

7. The apparatus of claim 6, wherein the filter is arranged around the oxygenator such that blood can move from the pump to the outlet without structural obstruction.

8. The apparatus of claim 1, wherein the heat exchanger is arranged around the pump such that blood can move from the pump to the heat exchanger without structural obstruction.

9. The apparatus of claim 1, wherein the oxygenator is arranged around the heat exchanger such that blood can move from the heat exchanger to the oxygenator without structural obstruction.

10. The apparatus of claim 1, wherein the pump is capable of delivering outflow over a substantially 360 degree perimeter.

11. The apparatus of claim 10, wherein the pump is a centrifugal pump or a diaphragm pump.

12. The apparatus of claim 1, wherein the pump comprises a central axis and is capable of delivering flow in a radially outward direction through substantially all of 360 degrees from the central axis.

13. The apparatus of claim 1, wherein the pump comprises a central axis and can pump blood radially outward to the heat exchanger in a substantially transverse direction to the central axis.

14. The apparatus of claim 13, wherein blood can move radially outward from the pump through substantially all of 360 degrees around the central axis.

15. The apparatus of claim 14, wherein blood can move radially outward from the heat exchanger through substantially all of 360 degrees around the central axis.

16. The apparatus of claim 15, wherein the pump has a central axis and blood can move radially outward from the oxygenator through substantially all of 360 degrees around the central axis.

17. The apparatus of claim 16 further comprising a filter, wherein blood can move radially outward from the filter through substantially all of 360 degrees around the central axis.

18. The apparatus of claim 1, wherein the plurality of heat transfer elements include a lumen through which a fluid medium can be supplied in order to control the temperature of blood moving between the heat transfer elements.

19. The apparatus of claim 18, wherein the plurality of heat transfer elements are arranged such that movement of the fluid medium through the plurality of heat transfer elements is substantially transverse to the radially outward direction that blood can move between the plurality of heat transfer elements.

20. The apparatus of claim 1, wherein the oxygenator comprises a plurality of gas exchange elements that include lumens through which an oxygen-containing gas medium can be supplied in order to oxygenate blood moving between the plurality of gas exchange elements.

21. The apparatus of claim 20, wherein the plurality of gas exchange elements are arranged such that movement of the gas medium through the plurality of gas exchange elements is substantially transverse to the radially outward direction that blood can move between the plurality of gas exchange elements.
